(19) United States
(12) Patent Application Publication
(10) Pub. No.: US 2014/0162295 A1
(43) Pub. Date: Jun. 12, 2014

(54) Method and Biomarker for Detecting of Acute Kidney Injury

(71) Applicant: NATIONAL TAIWAN UNIVERSITY, Taipei (TW)

(72) Inventors: Wen-Je KO, Taipei City (TW); Guang-Huar YOUNG, Taipei City (TW); Vin-Cent WU, Taipei City (TW); Tao-Min HUANG, Taipei City (TW)

(73) Assignee: NATIONAL TAIWAN UNIVERSITY, Taipei (TW)

(21) Appl. No.: 13/708,089

(22) Filed: Dec. 7, 2012

(51) Int. Cl. G01N 33/68; C07K 16/18; C07K 14/435

(52) U.S. Cl. CPC G01N 33/6893; C07K 14/435; C07K 16/18
USPC 435/7.92; 436/501; 435/7.1; 435/23; 530/350; 530/387.9

(57) ABSTRACT

A method and a biomarker for detecting of acute kidney injury is provided, wherein the method of the present comprises the following steps: detecting a soluble form of hemojuvelin in a sample obtained from a subject, wherein when the soluble form of hemojuvelin is present in the sample, the subject is identified as having acute kidney injury.

### BACKGROUND OF THE INVENTION

1. Field of the Invention

The present invention relates to a method and a biomarker for detecting of acute kidney injury (AKI) and, more particularly, to a method and a biomarker for detecting a soluble form of hemojuvelin in a urine sample from a subject to identify whether the subject is suffered from acute kidney injury in an early stage or not.

2. Description of Related Art

Acute kidney injury (AKI) is a serious complication in hospitals, resulting in a prolonged hospital stay and high mortality. Cardiac disease and cardiac surgery are both common causes of AKI. In order to provide early and suitable treatment to the subject with AKI, serum creatinine and RIFLE criteria are generally used to aid detection and assessment of AKI in the subject.

However, the expression of serum creatinine may be influenced by nutrition, infection and volume of body fluid and the elevated expression thereof may be not consistent with the dysfunction of tubular. Hence, serum creatinine as a biomarker for AKI is still has its limitations.

Besides serum creatinine, other biomarkers such as neutrophil gelatinase-associated lipocalin (NGAL), interleukin-18 (IL-18), kidney injury molecule-1 (KIM-1), L-type fatty acid binding protein (L-FABP) and Hepicidin-25 may also be used for AKI detection. However, these biomarkers also have their disadvantages. For example, a dialysis process has to be performed prior to detections with NGAL, IL-18 or KIM-1. In addition, the elevated expression of IL-18 and KIM-1 is not highly related to severities of AKI. Hence, there are no reliable biomarkers that can be used to detect AKI simply and rapidly.

Therefore, it is desirable to provide a novel biomarker to predict AKI in an early stage; and a novel method for detecting AKI that the sample pre-treatment can be simplified.

### SUMMARY OF THE INVENTION

An object of the present invention is to provide a method for detecting of acute kidney injury, which can identify whether a patient is suffered from the acute kidney injury in an early stage.

Another object of the present invention is to provide a biomarker and a kit for detecting of acute kidney injury. When the biomarker or the kit of the present invention is used, the level of the biomarker can be detected in a urine sample from a subject, so a non-invasive detection can be accomplished.

To achieve the object, the method for detecting of acute kidney injury in a subject of the present invention comprises the following steps: detecting a soluble form of hemojuvelin in a sample obtained from the subject, wherein when the soluble form of hemojuvelin is present in the sample, the subject is identified as having acute kidney injury.

In addition, the biomarker for detecting of acute kidney injury in a urine sample from a subject of the present invention can be a peptide fragment, a derivative of the peptide fragment, a mutation of the peptide fragment, or an antibody corresponding to the peptide fragment of a soluble form of hemojuvelin.

Furthermore, the kit for detecting of acute kidney injury in a urine sample from a subject of the present invention comprises: an anti-hemojuvelin antibody that specifically binds to one or more epitopes of a soluble form of hemojuvelin.

It is well known that AKI is a rapid loss of kidney function. Once patients have AKI, the mortality thereof is high. In order to provide early and suitable treatment to the subject with AKI, serum creatinine and RIFLE criteria are generally used to aid detection and assessment of AKI in the subject. However, the generally used biomarker and criteria still have their limitations. For example, when serum creatinine is used as a biomarker, the level thereof is easily influenced by conditions of the subjects such as infections. In this case, doctors cannot provide suitable treatment timely and the treatment on the subject may be delayed. In order to solve the aforementioned problems, the present invention provides a novel early biomarker for detecting of AKI, which can be used to assess whether a subject has AKI or not in an early stages. Especially, it is confirmed that the novel early biomarker, a soluble form of hemojuvelin of the present invention is present in the subject within 3 hr of AKI, so it is possible to detect AKI in an early stage when the biomarker of the present invention is applied. In addition, the biomarker of the present invention can be directly detected in a urine sample from the subject, so a non-invasive detection can be obtained. Furthermore, the inventors of the present invention also confirmed that the soluble form of hemojuvelin is only present when kidneys are damaged, so the soluble form of hemojuvelin is a reliable biomarker for detection AKI.

The method of the present invention may further comprise a step: detecting the soluble form of hemojuvelin in a control sample when the step of detecting the soluble form of hemojuvelin in the sample obtained from the subject is performed; and comparing a first level of the soluble form of hemojuvelin in the sample obtained from the subject and a second level of the soluble form of hemojuvelin in the control sample, wherein when the first level is elevated relative to the second level, the subject is identified as having acute kidney injury. Herein, the control sample can be selected based on the experiences of doctors. For example, the control sample is a sample obtained from another subject with healthy kidneys, a sample obtained from the subject before operation, or a standard. In the case that the control sample is a sample obtained from the subject before operation (i.e. pre-operative sample), it is possible to determine whether AKI is raised in the subject when the pre-operative sample from the subject and the post-operative sample there from are compared. In the case that the control sample is a sample obtained from another subject with healthy kidneys, it is possible to determine whether the subject is suffered from not only AKI but also chronic kidney disease. However, the present invention is not limited thereto.

In the method of the present invention, the sample and the control sample can be a urine sample, a blood sample, or a tissue sample respectively, but the types of the sample and the control sample should be the same. Preferably, both the sample and the control sample are serum samples or tissue samples. For the purpose of the non-invasive detection, a urine sample is preferable.

In addition, in the method of the present invention, the soluble form of hemojuvelin can be detected by any method generally used in the art. For example, the soluble form of hemojuvelin is detected by western blot analysis, electrophoresis, enzyme-linked immunosorbent assay (ELISA), immunohistochemistry (IHC), immunoprecipitation (IP), mass spectrometry (MS), real time polymerase chain reaction (RT-PCR) or real time quantitative polymerase chain reaction (real time Q-PCR). For the purpose of detecting the biomarker in the urine sample, western blot analysis, electrophoresis, ELISA, IHC, IP, or MS is more preferable. However, the present invention is not limited thereto.

The known sequence of full-length human hemojuvelin is represented by SEQ ID NO:1, that of mouse hemojuvelin is represented by SEQ ID NO:2, and that of rat hemojuvelin is represented by SEQ ID NO: 3. After the full length hemojuvelin is digested at a minus protease cutting site (i.e. a furin cutting site) 330-336, a soluble form of hemojuvelin is obtained. Hence, in the method and the biomarker of the present invention, the soluble form of hemojuvelin is a peptide fragment from amino acids I to 330 of hemojuvelin (minus protease cutting site, 330-336). Since the amino acids of the hemojuvelin may be deleted or mutated and the sequence thereof may be differed between different species, the soluble form of hemojuvelin may comprise an amino acid sequence having at least 90% identity to SEQ ID NO: 4, which is a peptide fragment from amino acids 1 to 330 of hemojuvelin represented by SEQ ID NO: 1. Preferably, the soluble form of hemojuvelin may comprise an amino acid sequence having at least 95% identity to SEQ ID NO: 4. More preferably, the soluble form of hemojuvelin comprises an amino acid sequence of SEQ ID NO: 4. SEQ ID NO: 1 (Bold: furin cutting site)
1 mgepgqspsp rsshgspptl stltlllllc ghahsqckil rcnaeyvsst lslrgggssg
61 alrggggggr gggvgsgglc ralrsyalct rrtartcrgd lafhsavhgi edlmiqhncs
121 rqgptapppp rgpalpgags glpapdpcdy egrfsrlhgr ppgflhcasf gdphvrsfhh
181 hfhtcrvqga wplldndflf vqatsspmal ganatatrkl tiifknmqec idqkvyqaev
241 dnlpvafedg singgdrpgg sslsiqtanp gnhveiqaay igttiiirqt agqlsfsikv
301 aedvamafsa eqdlqlcvgg cppsqrlsrs ernrrgaiti dtarrlckeg lpvedayfhs
361 cvfdvlisgd pnftvaaqaa ledaraflpd leklhlfpsd agvplssatl lapllsglfv
421 lwlciq

Furthermore, in the method and the biomarker of the present invention, the subject can be mammalians. Preferably, the subject is human.

In addition, in the kit of the present invention, the anti-hemojuvelin antibody can be prepared according to any conventional method generally used in the art, as long as the prepared anti-hemojuvelin antibody can specifically bind to one or more epitopes of a soluble form of hemojuvelin.

The term “acute kidney injury (AKI)” used in the present invention refers to any symptom of AKI without limitations to the AKI etiologies. For example, the AKI used in the present invention can be post-operative acute kidney injury, acute tubular necrosis-related injury, rhabdomyloysis with acute kidney injury (Rhabdo), chronic kidney disease (CKD)-related acute kidney injury, or urinary tract infection (UTI)-related acute kidney injury.

Other objects, advantages, and novel features of the invention will become more apparent from the following detailed description when taken in conjunction with the accompanying drawings.

### BRIEF DESCRIPTION OF THE DRAWINGS

FIG. 1A shows results of 2-dimentional electrophoresis analysis according to Experiment 1 of the present invention;

FIG. 1B shows results of 2-dimentional electrophoresis analysis and western blot according to Experiment 2 of the present invention;

FIGS. 2A-2C show results of western blot according to Experiment 3 of the present invention;

FIGS. 3A and 3B show results of western blot according to Experiment 4 of the present invention; and

FIGS. 4A and 4B show results of ELISA assays according to Experiment 5 of the present invention.

### DETAILED DESCRIPTION OF THE PREFERRED EMBODIMENT

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

### Human Specimens

All patients in the following experiments were registered in the National Taiwan University Hospital Study Group on Acute Renal Failure (NSARF). The AKI patients in the present embodiment were consecutively enrolled when AKI was diagnosed. The definition of AKI was based on the Acute Kidney Injury network (AKIN) criteria and has been validated in cardiac surgery patients for in-hospital mortality predictions. Paraffin-embedded human kidney sections were obtained from the NTUH Tissue Resource Center under and IRB-approved protocol.

Experiment 1—Comparison of Urinary Proteins from Pooled Urine Samples Obtained from Patients

In the present experiment, pooled urine samples from 15 healthy volunteers and 15 post-cardiac surgery patients with or without AKI were compared. The pooled urine samples were treated as follows.

The urine samples were collected in polypropylene tubes (BD biosciences) containing sodium azide (Sigma-Aldrich), and stored at −80° C. until use. The pooled urine samples were concentrated using an Amicon Ultra-15 centrifugal19 filter unit (Amersham Biosciences), passed through a PD-10 desalting column (Amersham Biosciences) and eluted with 10 mM PBS, pH 7.4. The eluted fractions containing proteins were added to two volumes of cold 20% TCA in acetone (Merck) for protein precipitation. The pellets were obtained by centrifugation at 10,000 g at 4° C. for 15 minutes. The pellets were vacuum dried on a SpeedVac (SC110).The treated urine samples (pellets) were stored at −20° C.

For the analysis with 2-dimentional electrophoresis (2-DE), a 1 mg aliquot of the pellet was rehydrated with 120 μl of 2-DE sample buffer (8 M urea, 2% Pharmalyte, pH3-10, 60 mM DTT, 0.5% Triton X-100, traces of bromophenol blue). Then, the obtained samples were run as the first dimension of the 2-DE on an 11 cm Immobiline DryStrip, pH 3-10 (GE Healthcare). Next, the proteins were separated in 12.5% SDS-PAGE gels for the second dimension, and then stained using Coomassie Brilliant Blue R250. For the mass spectrometric identification, each individual spot was cut out and digested with trypsin (Promega). The resulting peptides were extracted sequentially with 1% TFA and 0.1% TFA/50% ACN, and the pooled extracted were lyophilized and resuspended in 10 μL of 0.1% TFA. The peptides were characterized using a Qstar XL Q-TOF mass spectrometer (Applied Biosystems) coupled to an UltiMatc Nano LCsystem (Dionex/LC Packings). The protein identification generated from the MS/MS spectra were uploaded to the MASCOT search enginev2.2 (Matrix Science).

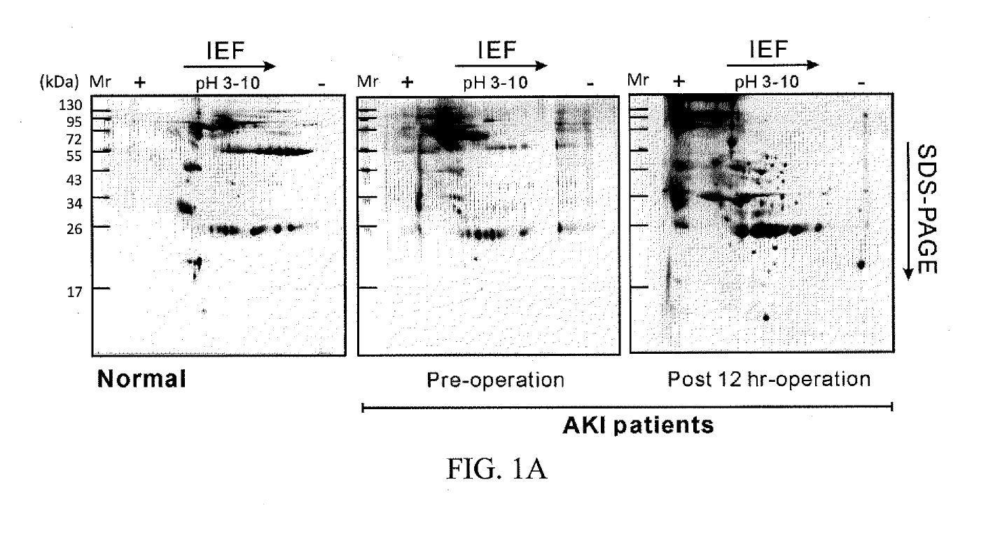

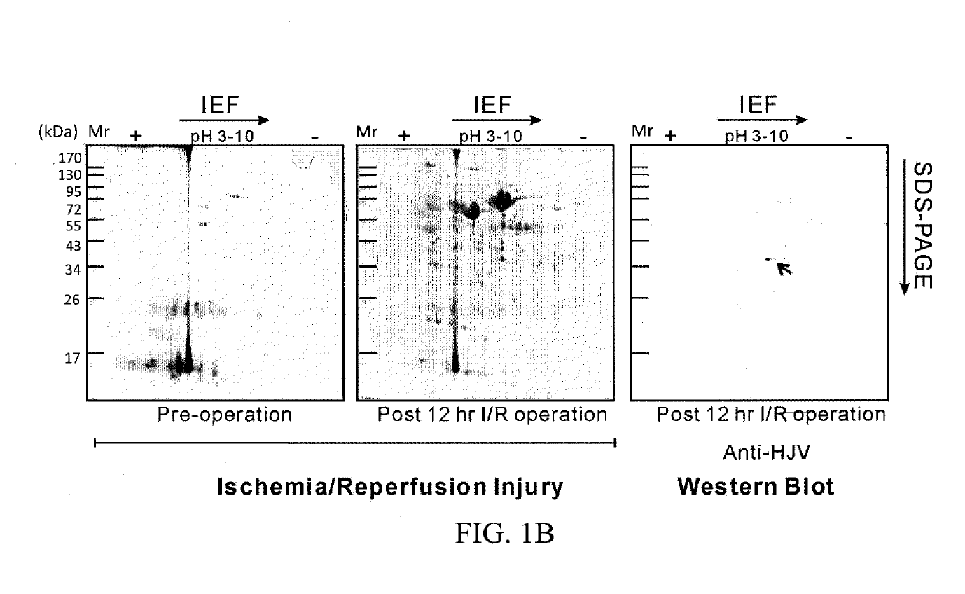

The results of 2-DE analysis of the present experiment are shown in FIG. 1A, wherein the left, middle and right panels show 2-DE maps of a urine pooled sample from a healthy volunteer, a urine pooled sample from an AKI patient pre-operation and a urine pooled sample from an AKI patient post 12 hr-operation respectively. Among the identified protein spots form post-operative 2-DE maps, the proteomic approach revealed that the levels of 25 proteins increased and the levels of 10 proteins decreased.

Experiment 2—Comparison of Urinary Proteins from Pooled Urine Samples Obtained from I/R Injury Rats

In order to confirm the types of the proteins with increased levels found in Experiment 1, pre- and post-unilateral renal ischemia/reperfusion (I/R), acute tubule necrosis (ATN) rats were examined in the present experiment. The models of I/R injured rats as AKI models were established as follows.

Male Wistar rats (110=15 g) or C57BL/6 mice (20±2 g) were briefly anesthetized with anesthesia cocktails (80 mg/kg ketamine plus 100 mg/kg xylazine) via i.p injection and were placed on a heating pad to maintain core temperatures of 37° C. The kidneys were exposed via an abdominal section; the left kidney was clamped by amicroaneurysm clip for 45 minutes, and the right kidney was removed. Reperfusion was confirmed visually upon release of the clamp. As a control, sham-operated animals were subjected to the same surgical procedure except the left renal pedicles were not clamped. The surgical wounds were closed, and the animals were given 1 ml of warm saline (i.p.) and remained in a warm incubator until they regained consciousness. The urine was collected by bladder massage and stored at −80° C. until use.

The urine samples of rats were treated with a procedure similar to that described in Experiment 1, so the detailed procedure is omitted herein. In addition, western blot was also performed on the urine samples collected from post 12 hr I/R rat. The procedure for western blot is shown as follows.

After protein separation, the gel was stained with Coomassie blue R250, transferred to a PVDF membrane, and detected using a polyclonal antibody against hemojuvelin (HJV), which was purchased from AVNOVA.

The results of 2-DE analysis and the western blot of the present experiment are shown in FIG. 1B, wherein the left and the middle panels show the 2-DE maps of pooled urinary proteins obtained from pre- and post-I/R injury rats respectively, and the right panel shows the western blot of pooled urinary proteins obtained from post-I/R injury rats. From the results of the 2-DE maps of the present experiment, of the urinary protein spots identified from the AKI patients and involved in iron metabolism (Experiment 1), only the protein detected by the anti-HJV antibody was up-regulated in the ATN mice. The other protein either could not be detected by the available antibodies or shifted in the direction opposite to that predicted by 2D-DIGE (data not shown). From the result of the western blot of the present experiment, a major 42 kDa urinary protein from ATN rats was identified with a specific anti-HJV antibody (indicated by an arrow).

The results of Experiments 1-2 show that the levels of this major urinary protein (a soluble form of HJV, sHJV) were increased in patients with AKI and post-I/R injured rats (AKI models). Hence, sHJV can be used as a biomarker for detect AKI.

In addition, according to the results of Experiments 1-2, it can be found that the expression of urinary proteins, especially sHJV protein obtained from the samples of pre-operation patients or rats is higher than that obtained from the samples of post-operation patient or rats. Hence, when the urine samples from a patient were collected before and after operation respectively, it is possible to detect whether the patient is suffered from AKI.

Experiment 3—Evaluation of Temporal Changes of HJV Correlated with Kidney Injury

The models of I/R injured rats established in Experiment 2 were used in the present experiment.

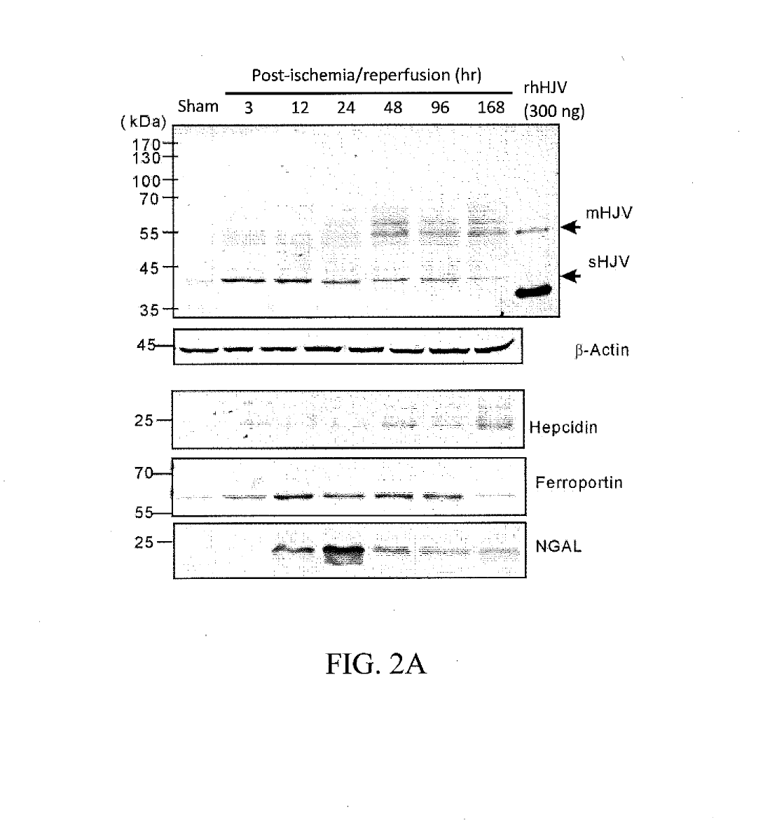

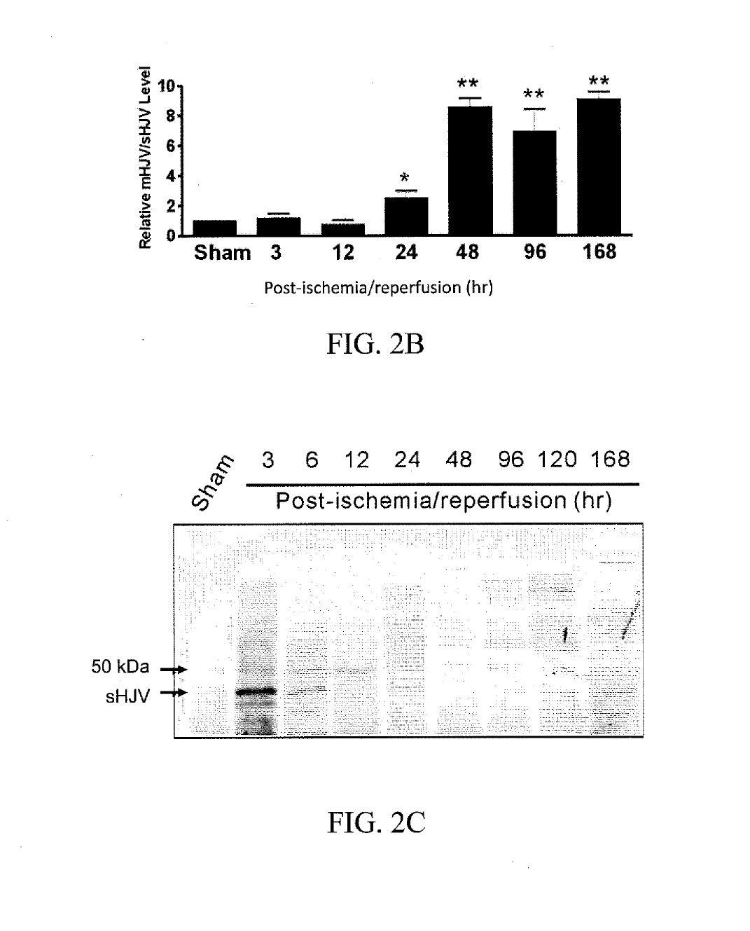

Kidney lysates obtained from pre- and post-I/R injured rats in RIPA buffer containing a protease inhibitor cocktail (Roche) were quantitated using the BCA protein assay reagent (Pierce Biotechnology). Next, a 30 μg sample of protein from each specimen at different time-points was separated using SDS-PAGE and transferred onto PVDF membranes (Millipore). The rabbit anti-HJV polyclonal antibody, rabbit anti-Ferroportin polyclonal antibody, mouse anti-HJV monoclonal antibody, and mouse anti-Hepcidin monoclonal antibody were purchased from AVNOVA. The mouse anti-β actin monoclonal antibody and the rabbit anti-NGAL polyclonal antibody were purchased from Santa Cruz. After the membrane was washed with PBST three times, the enzyme activity on the blot was visualized using NBT/BCIP (Promega), according to the manufacturer's instructions. In the present experiment, recombinant human HJV protein (rhHJV, R&D) was used as a control group. The results of the western blots of the present experiment are shown in FIG. 2A.

As shown in FIG. 2A, the level of a soluble form of HJV (sHJV) was rapidly increased at 3 hr, whereas that of the membrane-bound form of HJV (mHJV) increased gradually after 12 hr. However, the levels of other known biomarkers such as NGAL were elevated at 12 hr. These results indicate that the level of sHJV is creased more rapidly than those of other proteins. Furthermore, the relative mHJV/sHJV levels were quantified, and the quantification result is shown in FIG. 2B. The quantification result shows that sHJV is predominantly observed in the early stage, and mHJV is significantly observed in the late stage.

In addition, pooled urinary proteins obtained from pre- and post-I/R injured rats were also analyzed with western blot in the present experiment. The procedures for treating pooled urinary proteins and performing western blot are similar to those described above. The result is shown in FIG. 2C.

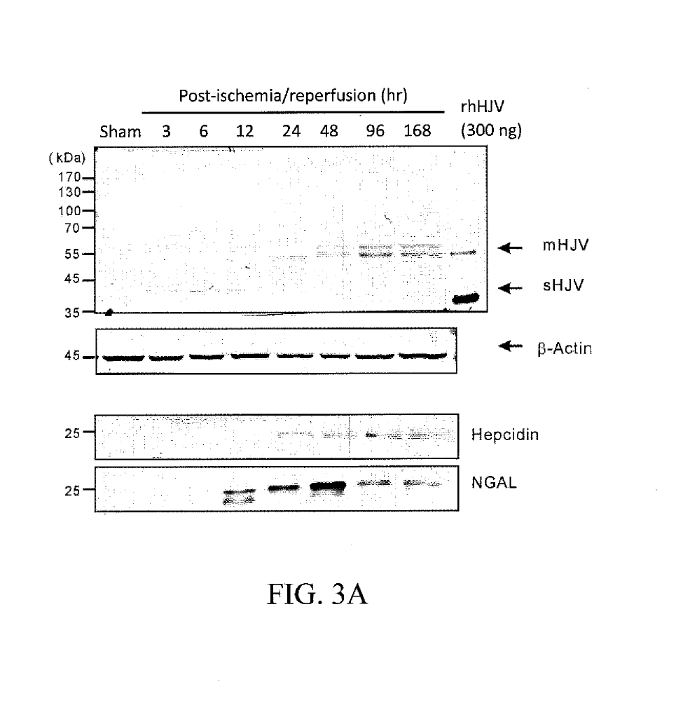

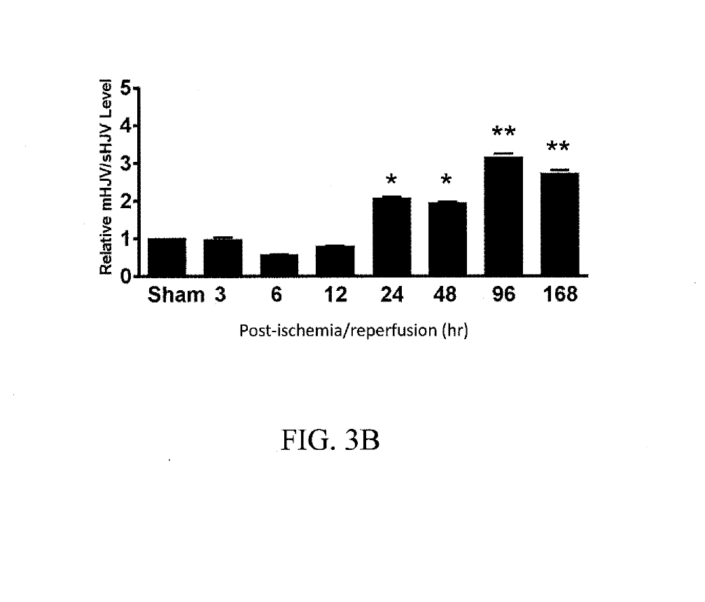

As shown in FIG. 2C, the result shows that urinary sHJV was predominantly observed, but less urinary mHJV was observed in the TCA-precipitated urinary proteins. In addition, the relative mHJV/sHJV levels were quantified, and the quantification result is shown in FIG. 3B. The quantification result shows that sHJV is predominantly observed in the early stage, and mHJV is significantly observed in the late stage.

According to the results shown in FIGS. 2A-2C, they indicate that sHJV can be used as an early biomarker for detecting AKI.

### Experiment 4—Evaluation of Temporal Changes of HJV in Glycerol-Induced ATN Rats

In the present experiment, rhabdomyolysis-associated ATN models were used, which were established by glycerol induction.

First, ATN was induced in male C57BL/6 mice by an i.m. injection with 50% glycerol (Amresco) solution in water (10 ml/kg body/wt). In brief, the animals (body weight 20±2 g) were injected with glycerol into the left and right hind femoral muscles. The animals received 160±10 μl of 50% glycerol/g body weight on average.

Next, pooled urinary proteins obtained from glycerol-induced ATN mice were analyzed with western blot, and the procedure for performing western blot is similar to those described above. The result is shown in FIG. 3A.

As shown in FIG. 3A, the expression of sHJV and mHJV proteins was observed prior to that of the kidney injury marker NGAL in mouse kidneys after injury. Especially, the sHJV was rapidly increased at 3 hr, whereas that of the membrane-bound form of HJV (mHJV) increased gradually after 12 hr.

The results shown in FIGS. 3A and 3B are consistent with those observed in the previous experiment. Hence, compared to other markers including NGAL, sHJV can be used as an early biomarker for detecting AKI.

Experiment 5—Evaluation of Temporal Changes of HJV in Patients with Different Disease Etiologies

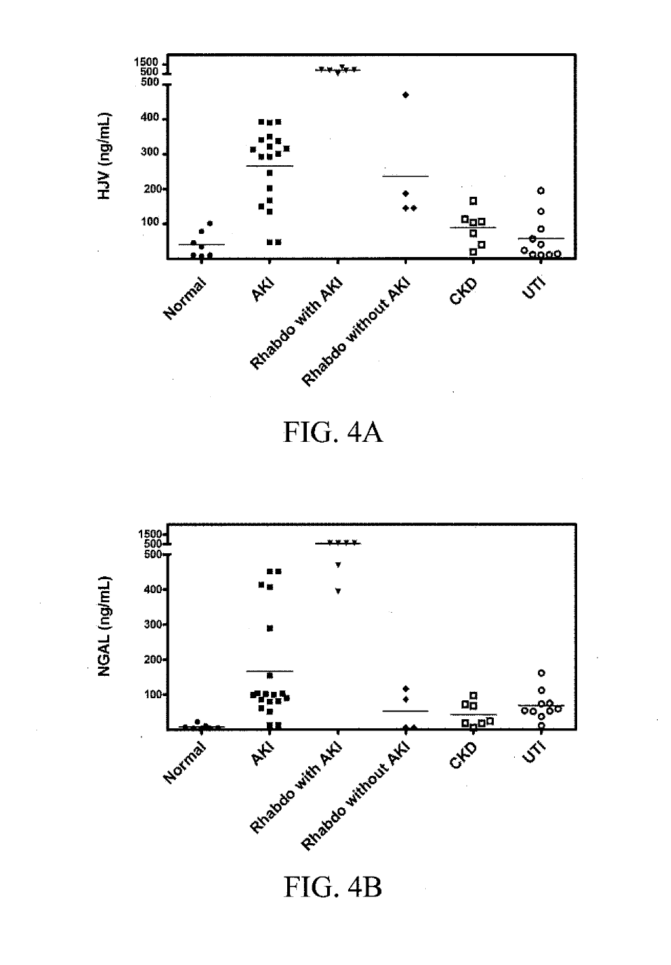

The present experiment is to examine whether urinary sHJV expression could stratify disease etiologies. Herein, 19 patients (Scr 4.11±2.81 mg/dl) from cardiac surgery (i.e. post-operative AKI, represented as AKI in the FIGS. 4A and 4B), 6 rhabdomyolysis patients with AKI (Scr 2.98±1.61 mg/dl) (represented as Rhabdo with AKI in the FIGS. 4A and 4B), 4rhabdomyolysis patients without AKI (Scr 0.88±0.15 mg/dl) (represented as Rhabdo without AKI in the FIGS. 4A and 4B), 7 patients with established chronic kidney disease (CKD) (Scr 2.51±1.15 mg/dl) (represented as CKD in the FIGS. 4A and 4B), 10 urinary tractinfection (UTI) patients (Scr 1.45±1.27 mg/dl) (represented as UTI in the FIGS. 4A and 4B), and 7 healthy volunteers (Scr, 0.7±0.07 mg/dl) (represented as Normal in the FIGS. 4A and 4B) were analyzed. Herein, an ELISA assay was performed in the present experiment.

The procedure for performing the ELISA assay is shown as follows. The urine samples collected in separate polypropylene tubes containing sodium azide were stored at −80° C. until use. Each specimen was centrifuged at 800 g at 4° C. for 5 min, and the supernatant was removed. The urinary NGAL level was determined using a human lipocalin-2/NGAL ELISA kit (R&D Systems), and the HJV level was determined using a human hemojuvelin ELISA kit (USCN). All operations were performed according to the manufacturer's protocols, and each assay was performed in duplicate. The results are shown in FIGS. 4A and 4B, wherein FIG. 4A is a result that urine samples were validated using NGAL, and FIG. 4B is a result that urine samples were validated using HJV.

As shown in FIGS. 4A and 4B, both the urinary HJV and NGAL concentrations in the rhabdomyolysis patients with AKI (898.01±213.03 ng/ml; n=6; represented as Rhabdo with AKI in the figure) and those in the post-operative AKI group (265.8±109.6 ng/ml; n=19; represented as AKI in the figure) were higher than those of the healthy volunteers (41.5±36.7 ng/ml; n=7; represent as normal in the figure), the CKD patients (88.68±49.22 ng/ml; n=7; represented as CKD in the figure), the UTI patients (58.45±62.8 ng/ml; n=10; represented as UTI in the figure) and the rhabdomyolysis patients without AKI (236.9±156.7 ng/ml; n=4; represented as Rhabdo without AKI in the figure). These results indicate that the urinary HJV and NGAL levels were elevated in AKI patients. Especially, the result that urine samples were validated using HJV is consistent with that using NGAL. Hence, HJV can be used as a biomarker for detecting AKI.

In addition, according to the results of the present experiment, it can be found that the expression HJV in patients with AKI is much higher than that in another subject with healthy kidneys (i.e. healthy volunteer in the present experiment). Hence, the urinary samples from healthy volunteer can be used as control samples. When a first level of the HJV in the urinary sample obtained from a tester (or a patient) is elevated relative to a second level of the HJV in the urinary sample obtained from a healthy volunteer, it indicate that the tester may have AKI.

Although both sHJV and mHJV may be detected in the present experiment, a person skilled in the art may understand that the expression of sHJV is present prior to that of mHJV, according to the results of the foregoing experiments. Hence, sHJV is a reliable biomarker for detecting AKI, especially detecting AKI in early stages such as few hours after operation.

In addition, in foregoing experiments, an anti-HJV antibody is used to detect sHJV in urinary samples. Hence, a person skilled in the art can understand that any anti-HJV antibody such as monoclonal and polyclonal antibody against HJV may be used for detecting AKI, as long as the antibody can specifically bind to one or more epitopes of a soluble form of HJV.

Although the present invention has been explained in relation to its preferred embodiment, it is to be understood that many other possible modifications and variations can be made without departing from the spirit and scope of the invention as hereinafter claimed.

1. A method for detecting of acute kidney injury in a subject, comprising the following steps:
providing a sample obtained from a subject and a sample, wherein the sample and the control sample are urine samples, and the control sample is a sample obtained from another subject with healthy kidneys, a sample obtained from the subject before operation, or a standard;
detecting a soluble form of hemojuvelin in a sample obtained from the subject and the control sample; and
comparing a first level of the soluble form of hemojuvelin in the sample obtained from the subject and a second level of the soluble form of hemojuvelin in the control sample, wherein when the first level is elevated relative to the second level, the subject is identified as having acute kidney injury.

2-7. (canceled)

8. The method as claimed in claim 1, wherein the soluble form of hemojuvelin is detected by western blot analysis, electrophoresis, enzyme-linked immunosorbent assay (ELISA), immunohistochemistry (IHC), immunoprecipitation (IP) or mass spectrometry (MS).

9. The method as claimed in claim 1, wherein the soluble form of hemojuvelin is a peptide fragment from amino acids 1 to 330 of hemojuvelin.

10. (canceled)

11. The method as claimed in claim 1, wherein the soluble form of hemojuvelin comprises an amino acid sequence of SEQ ID NO: 4.

12. The method as claimed in claim 1, wherein the acute kidney injury is post-operative acute kidney injury, acute tubular necrosis-related injury, or rhabdomyloysis with acute kidney injury.

13. A biomarker for detecting of acute kidney injury in a urine sample from a subject, which is a peptide fragment, a derivative of the peptide fragment, a mutation of the peptide fragment, or an antibody corresponding to the peptide fragment of a soluble form of hemojuvelin.

14. The biomarker as claimed in claim 13, wherein the peptide fragment is a peptide fragment from amino acids 1 to 330 of hemojuvelin.

15. The biomarker as claimed in claim 13, wherein the peptide fragment comprises an amino acid sequence having at least 90% identity to SEQ ID NO: 4.

16. The biomarker as claimed in claim 15, wherein the peptide fragment comprises an amino acid sequence of SEQ ID NO: 4.


SEQUENCE LISTING (4 sequences)

SEQ ID NO: 1 - protein, 426 aa, Homo sapiens
  ref <308>: DATABASE ACCESSION NUMBER: NCBI/NP_998818.1
  ref <309>: DATABASE ENTRY DATE: 2012-12-06
  ref <313>: RELEVANT RESIDUES IN SEQ ID NO: (1)..(426)
MGEPGQSPSPRSSHGSPPTLSTLTLLLLLCGHAHSQCKILRCNAEYVSSTLSLRGGGSSGALRGGGGGGRGGGVGSGGLCRALRSYALCTRRTARTCRGDLAFHSAVHGIEDLMIQHNCSRQGPTAPPPPRGPALPGAGSGLPAPDPCDYEGRFSRLHGRPPGFLHCASFGDPHVRSFHHHFHTCRVQGAWPLLDNDFLFVQATSSPMALGANATATRKLTIIFKNMQECIDQKVYQAEVDNLPVAFEDGSINGGDRPGGSSLSIQTANPGNHVEIQAAYIGTTIIIRQTAGQLSFSIKVAEDVAMAFSAEQDLQLCVGGCPPSQRLSRSERNRRGAITIDTARRLCKEGLPVEDAYFHSCVFDVLISGDPNFTVAAQAALEDARAFLPDLEKLHLFPSDAGVPLSSATLLAPLLSGLFVLWLCIQ

SEQ ID NO: 2 - protein, 420 aa, Mus musculus
  ref <308>: DATABASE ACCESSION NUMBER: NCBI/NP_081402.3
  ref <309>: DATABASE ENTRY DATE: 2012-12-06
  ref <313>: RELEVANT RESIDUES IN SEQ ID NO: (1)..(420)
MGQSPSPRSPHGSPPTLSTLTLLLLLCGQAHSQCKILRCNAEYVSSTLSLRGGGSPDTPRGGGRGGLASGGLCRALRSYALCTRRTARTCRGDLAFHSAVHGIEDLMIQHNCSRQGPTAPPPARGPALPGAGPAPLTPDPCDYEARFSRLHGRAPGFLHCASFGDPHVRSFHNQFHTCRVQGAWPLLDNDFLFVQATSSPVSSGANATTIRKITIIFKNMQECIDQKVYQAEVDNLPAAFEDGSINGGDRPGGSSLSIQTANLGSHVEIRAAYIGTTIIIRQTAGQLSFSIRVAEDVARAFSAEQDLQLCVGGCPPSQRLSRSERNRRGAIAIDTARRLCKEGLPVEDAYFQSCVFDVSVSGDPNFTVAAQTALDDARIFLTDLENLHLFPSDAGPPLSPAICLVPLLSALFVLWLCFSK

SEQ ID NO: 3 - protein, 422 aa, Rattus norvegicus
  ref <308>: DATABASE ACCESSION NUMBER: NCBI/NP_001012080.1
  ref <309>: DATABASE ENTRY DATE: 2012-12-06
  ref <313>: RELEVANT RESIDUES IN SEQ ID NO: (1)..(422)
MGDRGRSPSLRSPHGSPPTLSTLTLLLLLCGQAHSQCKILRCNAEYVSSTLSLRGGGSPDTPHGGGRGGPASGGLCRALRSYALCTRRTARTCRGDLAFHSAVHGIEDLMIQHNCSRQGPTASPPARGPALPGAGPAPLTPDPCDYEARFSRLHGRTPGFLHCASFGDPHVRSFHNHFHTCRVQGAWPLLDNDFLFVQATSSPVASGANATTIRKITIIFKNMQECIDQKVYQAEVDNLPAAFEDGSVNGGDRPGGSSLSIQTANLGSHVEIRAAYIGTTIIVRQTAGQLSFSIRVAEDVARAFSAEQDLQLCVGGCPPSQRLSRSERNRRGAIAIDTARRLCKEGLPVEDAYFQSCVFDVSVSGDPNFTVAAQSALDDARVFLTDLENLHLFPVDAGPPLSPATCLVRLLSVLFVLWFCIQ

SEQ ID NO: 4 - protein, 330 aa, Homo sapiens
  ref <308>: DATABASE ACCESSION NUMBER: NCBI/NP_998818.1
  ref <309>: DATABASE ENTRY DATE: 2012-12-06
  ref <313>: RELEVANT RESIDUES IN SEQ ID NO: (1)..(330)
MGEPGQSPSPRSSHGSPPTLSTLTLLLLLCGHAHSQCKILRCNAEYVSSTLSLRGGGSSGALRGGGGGGRGGGVGSGGLCRALRSYALCTRRTARTCRGDLAFHSAVHGIEDLMIQHNCSRQGPTAPPPPRGPALPGAGSGLPAPDPCDYEGRFSRLHGRPPGFLHCASFGDPHVRSFHHHFHTCRVQGAWPLLDNDFLFVQATSSPMALGANATATRKLTIIFKNMQECIDQKVYQAEVDNLPVAFEDGSINGGDRPGGSSLSIQTANPGNHVEIQAAYIGTTIIIRQTAGQLSFSIKVAEDVAMAFSAEQDLQLCVGGCPPSQRLSRS